(19) United States
(12) Patent Application Publication
(10) Pub. No.: US 2022/0238034 A1
(43) Pub. Date: Jul. 28, 2022

(54) DYNAMIC ASSESSMENT AND ADJUSTMENT OF PC USER WELLBEING

(71) Applicant: Dell Products L.P., Round Rock, TX (US)

(72) Inventors: Rex BRYAN, Round Rock, TX (US); Karun REDDY, Austin, TX (US); Syed AHMED, Austin, TX (US)

(73) Assignee: Dell Products L.P., Round Rock, TX (US)

(21) Appl. No.: 17/160,362

(22) Filed: Jan. 27, 2021

(51) Int. Cl. G09B 19/00; G16H 50/30

(52) U.S. Cl. CPC G09B 19/00; G06N 20/00; G16H 50/30

(57) ABSTRACT

A disclosed method for assessing and influencing behavior, including workplace activity, of a personal computer user. User behavior and/or activity information pertaining to the user is received from the user's various smart devices such as a laptop, desktop, or hybrid computer, a mobile device, and a wearable device. The user's information is collected and an intelligence resource learns norms and distributions for various parameters and defines one or more user behavior thresholds. The user's present condition may be evaluated based on recently observed and collected activity and a risk factor score may be assigned. An initial behavior-influencing action appropriate for the user condition and risk level is performed. Disclosed methods further include performing at least one extended action pertaining to the initial action. The extended action can include evaluating and effectiveness of the initial action or the performance of a progressive action or a supplementary action.

### TECHNICAL FIELD

The present disclosure relates to healthcare and, more specifically, the wellbeing of occupational PC users.

### BACKGROUND

As the value and use of information continues to increase, individuals and businesses seek additional ways to process and store information. One option available to users is information handling systems. An information handling system generally processes, compiles, stores, and/or communicates information or data for business, personal, or other purposes thereby allowing users to take advantage of the value of the information. Because technology and information handling needs and requirements vary between different users or applications, information handling systems may also vary regarding what information is handled, how the information is handled, how much information is processed, stored, or communicated, and how quickly and efficiently the information may be processed, stored, or communicated. The variations in information handling systems allow for information handling systems to be general or configured for a specific user or specific use such as financial transaction processing, airline reservations, enterprise data storage, or global communications. In addition, information handling systems may include a variety of hardware and software components that may be configured to process, store, and communicate information and may include one or more computer systems, data storage systems, and networking systems.

Traditionally, the use of end-user computing devices and other information handling systems within the workplace to improve wellness has been less than comprehensive. While employers have recognized the benefits of employee wellness for decades for both reducing medical and absentee costs and as a tool for employee retention, attraction and productivity, employer-sanctioned wellness programs have frequently emphasized training and education, enticement programs (steps challenges, gym memberships), and office ergonomics programs for the design and selection of office space, furniture and workstation setup.

More recently, software-based health and safety services have been deployed to facilitate training, ergonomics evaluations, and work monitoring, but the actions taken by these programs has been largely limited to sending simplistic, fixed-content messages, such as break reminders and posture prompts, at fixed and periodic intervals with little or no awareness of the user's current, recent, and scheduled activities. A wellness prompt arriving in the middle of a focused and productive work session is as likely to disrupt productivity as it is to improve wellness. Similarly, a break reminder arriving moments after a user returns from lunch is of little value and may even be harmful to the extent that the reminder establishes or reinforces skepticism and a belief that such prompts and programs, however well intentioned, are not generally helpful or effective.

### SUMMARY

In accordance with teachings disclosed herein, common problems associated with traditional corporate wellness initiatives are addressed, in whole or part, by one or more disclosed systems and methods for assessing and influencing user behavior to improve user wellbeing using a heterogeneous combination of information handling system types, sensor-based functionality, often pre-existing and embedded, and highly available cloud-based intelligence and storage. In addition to leveraging a diverse set of resources to generate and perform at least one timely and effective behavior-influencing action (behavior-influencing action), disclosed methods include extended functionality, i.e., functionality performed after an initial behavior-influencing action is performed. Extended functionality disclosed herein includes, as non limiting examples, functionality for sensing the effectiveness of an any particular message or prompt and passing effectiveness data back to intelligence resource(s), functionality for a sequence of two or more progressively assertive or noticeable actions until such time as the user changes behavior; and functionality to provide supplementary actions to other parties for assistance in user behavior change and risk reduction.

Information handling systems suitable for use in implementing disclosed subject matter may include one or more of any of the following resources and services: input devices configured to collect user wellness information; biometric sensing devices and/or device interfaces; environmental sensing devices and/or device interfaces; cloud aggregated public data such as weather, air quality index (AQI), cloud based storage and intelligence engines for housing, analyzing, learning, and drawing conclusions from historic user behavior data; resources for collecting hardware and software usage data; cloud-based service to aggregate, clean, and prioritize data sources to create a holistic view of user wellbeing risk factors; cloud services to generate a dynamic intervention action, to reduce the user's risk, and pass the intervention action through an IHS to an end user device such as by displaying an alert, actuating an output device, slowing performance, or recommending a specific action; and a cloud service configured to push intervention action to other devices such as smart watch, phone, dock, display.

One or more methods and systems disclosed herein beneficially employ a cloud-based data aggregation and intelligence module or service to extract a prioritized, holistic view of user risk factors across devices, environments, and activities i.e. wearables, mobile phones, PC sensors, app data such as calendar events, building sensors, and environmental data. Disclosed intelligence modules may execute an algorithm to continually assess wellbeing risk factors including, without limitation, posture, activity level, eye fatigue, head pose, air temperature, humidity, and quality, and noise exposure among others, compare to established threshold values and historic norms and dynamically provide user feedback and behavior modification actions to improve wellbeing. Some embodiments may use sensor data such as non-intrusive low power vision-based solutions to assess the user response effectiveness of intervention prompts. An algorithm may learn user response to prompts and to adjust interventions to subtly encourage individualized long-term behavior modification without being disruptive.

The number and variety of parameters that may be aggregated is expansive. Wearables and mobile devices may provide vital sign data such as heart rate data, including resting, average, and instantaneous heart rate data, heart rate variation, theoretical maximum heartrate (TMH), and heart rate intensity (HR/TMH), body temperature, blood pressure, blood oxygen level, Galvanic skin response, and the like. These devices may further provide environmental and positional data indicative of GPS location, BT proximity, motion, including velocity and compass direction, air temperature, sunlight exposure levels, and so forth. Environmental sensors either embedded in the PC or in externally connected devices may report data indicative of light, motion, carbon monoxide (CO), carbon dioxide, CO₂, air temperature, VOC data, particulate counts, including mold and pollen counts and types, humidity, environmental noise, etc.)

Cloud based resources may provide, as examples, weather data, air quality index (AQI) data, IP location, historic norms, employer provided building data, etc.) PCs may provide keyboard and/or other input device activity, proximity/presence, motion, location, application usage data, camera based posture detection/estimation, head pose, gaze point, facial recognition and mood detection, radar, lidar, and so forth.) In addition, externally connected sit/stand desk controllers, PC docking stations, display monitors, chair sensor, may all provide additional inputs.

In one respect, subject matter included herein discloses a method for assessing and influencing behavior, including workplace behavior, of a personal computer user. In this context, behavior refers to physical behavior including activities performed by the user and/or the lack thereof, i.e., inactivity, as well as vital sign information and/or environmental information associated with such physical activity. Behavior information pertaining to the user is received from the user's one or more of the user's various smart devices such as a laptop, desktop, or hybrid computer, a mobile device, and a wearable device such as an activity tracker, smart watch, or the like. The behavior information may include The user's behavior information is collected and additional parameters may be derived from the collected information. An intelligence resource such as a cloud based artificial intelligence resource learns norms and distributions for various parameters and defines one or more user behavior thresholds. The user's present condition may be evaluated based on recently collected activity and a risk factor score may be assigned. An initial behavior-influencing action, appropriate for the user condition and risk level is performed. Disclosed methods further include performing at least one extended action pertaining to the initial action. The extended action can include evaluating and effectiveness of the initial action or the performance of a progressive action or a supplementary action as described in more detail below.

Technical advantages of the present disclosure may be apparent to those of ordinary skill in the art in view of the following specification, claims, and drawings.

### BRIEF DESCRIPTION OF THE DRAWINGS

A more complete understanding of the present embodiments and advantages thereof may be acquired by referring to the following description taken in conjunction with the accompanying drawings, in which like reference numbers indicate like features, and wherein:

FIG. 1 illustrates a platform for assessing and adjusting PC user wellbeing;

FIG. 2 illustrates an exemplary information handling system suitable for use in conjunction with disclosed subject matter;

FIG. 3 illustrates elements of a method for assessing and adjusting user wellbeing; and

FIG. 4 illustrates a detailed implementation of a wellbeing assessment and adjustment method.

### DETAILED DESCRIPTION

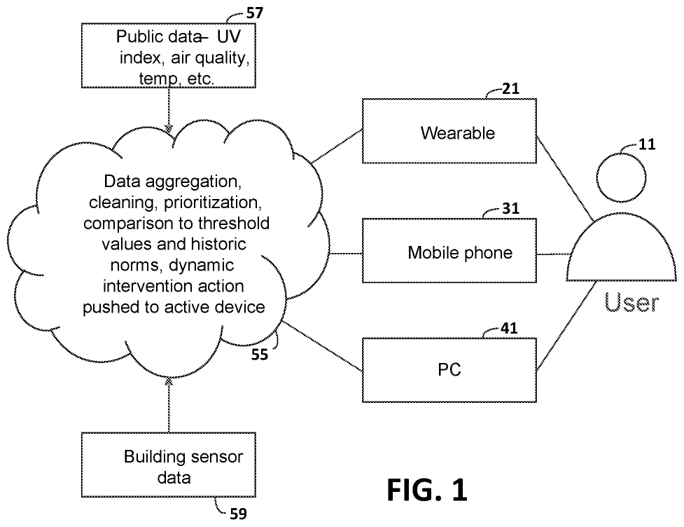

Preferred embodiments and their advantages are best understood by reference to FIGS. 1-4, wherein like numbers are used to indicate like and corresponding parts.

For the purposes of this disclosure, an information handling system may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, entertainment, or other purposes. For example, an information handling system may be a personal computer, a personal digital assistant (PDA), a consumer electronic device, a network data storage device, or any other suitable device and may vary in size, shape, performance, functionality, and price. The information handling system may include memory, one or more processing resources such as a central processing unit (CPU) or hardware or software control logic. Additional components of the information handling system may include one or more data storage devices, one or more communications ports for communicating with external devices as well as various input and output (I/O) devices, such as a keyboard, a mouse, and a video display. The information handling system may also include one or more buses operable to transmit communication between the various hardware components.

In this disclosure, the term “information handling resource” may broadly refer to any component system, device or apparatus of an information handling system, including without limitation processors, buses, memories, input-output devices and/or interfaces, storage resources, network interfaces, motherboards, electro-mechanical devices (e.g., fans), displays, and power supplies.

Referring now to the drawings, FIG. 1 depicts a platform for the collection of data associated with a user 101 whose job function includes significant interaction with one or more information handling systems categorized and referred to herein as personal computers (PCs). Although the PC acronym has been known and used in the industry since the early 1980's, the term as used herein is intended to emphasize information handling systems such as a desktop devices and laptop devices attached to a docking station, both of which generally include a traditional keyboard, mouse, and monitor configuration and both of which are typically accessed by a user in a fixed position, such as seated in an ergonomic chair, but increasingly also including users who prefer to stand while working at a PC. In addition, at least some aspects of disclosed methods for assessing PC user wellbeing may be particularly germane and beneficial for users who spend a significant amount of their working day on some form of PC and for corporations and other entities that employ large numbers of such employees.

FIG. 1 illustrates a user 11 of a PC 41 sourcing data to the PC 41 and to other smart devices associated with user 11, including a mobile phone 31 and a wearable technology device referred to herein simply as wearable 21. The user data acquired by these devices includes direct vital sign data such as heart rate, etc. as well as information indicative of the user's physical behavior while working on PC 41. The platform illustrated in FIG. 1 is unique and highly beneficial in its combined use of the user's various smart devices as the gatherers of user behavior information and particularly in its inclusion of the PC 41 as a participating component within a wellbeing initiative. The PC 41 is significant within the context of the wellbeing methods disclosed herein at least because PCs such as PC 41 are widely used in connection with jobs that require the user to be at the PC for all or most of the day. Accordingly, PC usage may correlate with ailments and issues stemming from prolonged, fixed-position activity and inactivity. In addition, although PCs have not been widely employed for health and wellness data collection, laptop and desktop devices have increasingly incorporated sensor-based features and functions that can be leveraged to provide wellness-relevant information that may not be attainable from other sources. More specifically, the type of user activity or non activity the user engages in while working on PC 41 is qualitatively distinct from using a mobile device or a wearable, which is not actively “used” at all.

Returning to FIG. 1, user information acquired by wearable 21, mobile phone 31, and PC 41 is forwarded to a cloud based intelligence resource 55, where the user data is aggregated and analyzed. In at least some embodiments, the intelligence resource 55 is provisioned with processing, storage, and analytical resources to discover and acquire an understanding of the user behavior data including historic norms and thresholds. In addition, intelligence resource 55 is configured to map each of the acquired parameters against each of the other one or more parameters to identify dependencies that correlate with a parameter of interest. Such dependency information may be used to identify behavior that needs to be cited.

The intelligence resource 55 illustrated in FIG. 1 is further configured to evaluate the user's present state or condition based on a comparison of recently acquired user data vs historically developed norms and thresholds. An action intended to alert the user and influence the user's future behavior or activity, such as a push notification, may be initiated.

The platform 10 illustrated in FIG. 1 includes additional sources of data provided to intelligence resources 55. A public data source 57 may provide, as non limiting examples, UV index data, air quality data and temperature data, etc. In addition, the platform 11 illustrated in FIG. 1 includes a data source 59 comprising one or more sensors within the applicable building. Such sensors could provide information pertaining to the user presence and activity level when not present in front of PC 41.

While platform 11 is illustrated with a particular combination of data sources and smart devices, the illustration is exemplary and other additional sources of data and other combinations of smart devices will be readily apparent to those of ordinary skill having the benefit of this disclosure.

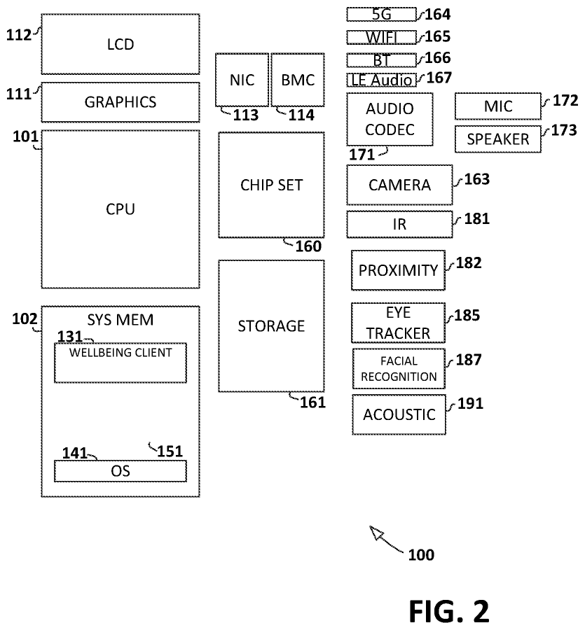

Referring now to FIG. 2, a block diagram of an information handling system 100 suitable for use as PC 41 in the platform 11 of FIG. 1 is depicted. Although the information handling system 100 illustrated in FIG. 2 includes elements that may be associated with a laptop or desktop computer, disclosed wellbeing assessment and adjustment features may be beneficially included in other types of information handling systems and those of ordinary skill in the field of electronic devices will readily appreciate that the depicted system is exemplary and that other devices, not explicitly illustrated in FIG. 2, including smart phones, tablets, hybrid devices, and dedicated video/conferencing devices. It will be further appreciated that, for the sake of clarity and brevity, many elements and components of information handling system 100 have been omitted from the depiction in FIG. 2.

The information handling system 100 illustrated in FIG. 2 includes a general purpose processor or central processing unit (CPU) 101 communicatively coupled to various peripheral devices generically referred to herein as information handling resources. The information handling resources illustrated in FIG. 2 include a system memory 102 suitable for storing data (not explicitly depicted in FIG. 1) intended for and/or generated by CPU 101 as well as computer executable instructions, sometimes referred to as programs, applications, and the like, for performing specific tasks and functions.

The programs residing in the system memory 102 illustrated in FIG. 2 include an operating system 141 which manages system resources and provides a functional platform for CPU 101 to execute application programs. The applications programs residing in the system memory 102 illustrated in FIG. 2 include a wellbeing client application 131, which may be configured to communicate with intelligence resource 55 and to coordinate the delivery of user data to intelligence resources 55. For the sake of clarity, many other programs executed by CPU 101, all or portions of which may be stored in system memory 102, are omitted from FIG. 2. In at least one embodiment, wellbeing client 131 monitors inputs from one or more sensors and one or more machine learning engines that may be germane to wellbeing assessment and adjustment.

The information handling system 100 of FIG. 2 further includes a graphics module 111 to process video information and render video on a display device such as the liquid crystal display (LCD) 112 and a chipset 120 to communicatively couple various peripheral devices to CPU 101. A network interface card (NIC) 113 supports Ethernet or another suitable broadband network connection. A baseboard management controller (BMC) 114 facilitates and supports external management of information handling system 100. The peripheral devices coupled to CPU 101 via chip set 160 include storage resources 161, camera 163, radio/transceiver resources 164-167 supporting various wireless communication transports and protocols, an audio codec 171 coupled to a microphone 172 and speakers 173, and an IR transceiver 181.

The information handling system 100 illustrated in FIG. 2 includes one or more sensors and/or sensed functions which may be utilized by orchestrator 151. The sensed functions illustrated in FIG. 2 include a proximity detector 182, eye tracker 185, facial recognition module 187, and an acoustic resource 191. Eye tracker 185 may support monitoring and determination of eye position, gaze direction, and head pose, as non-limiting examples. Acoustic modules 191 may be used in conjunction with microphone 172 to measure the spectral content and other characteristics of a talker and determine the echo characteristics of a room or environment by, for example, determining an RT60 value or another reverberation time parameter for a room.

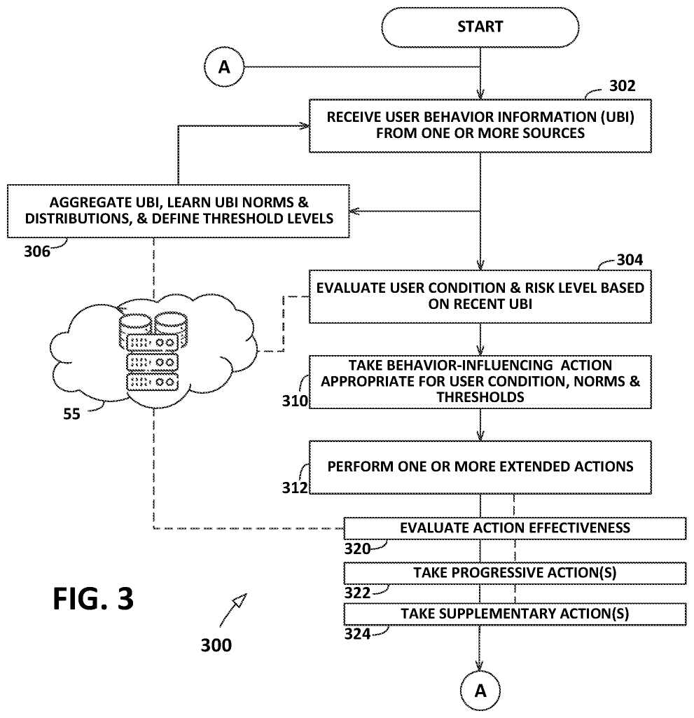

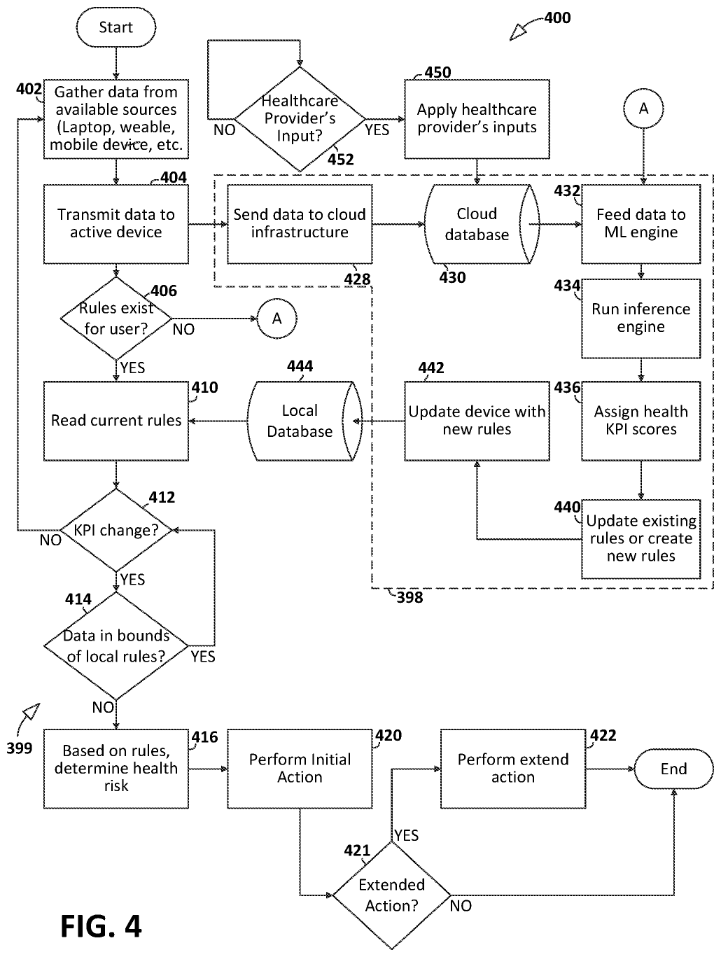

Referring now to FIG. 3 a method of assessing and influencing the wellbeing of a PC user is illustrated in flow diagram form. The illustrated method 300 includes receiving (302) user behavior information from one or more sources as previously described. The received information is aggregated (306), typically by an intelligence engine 55, which learns norms and defines threshold levels for the information. In one example, the intelligence engine 55 includes a machine learning engine that develops a user data knowledge base using one or more machine learning algorithms. An inference engine may then apply rules to the knowledge base to determine applicable threshold values for certain parameters as described in more detail in FIG. 4.

As depicted in FIG. 3, operations within method 300 access one or more functions, modules, engines, or resources generically referred to herein as intelligence resource 55. In at least some embodiments, the condition of a user and an associated risk level may be determined based on received information, provided at least in part by the user's smart devices, regarding habits, acdtivities, and/or behaviors of the PC user using artificial intelligence and/or machine learning algorithms, such as supervised, unsupervised, or reinforcement training algorithms, to analyze the usage data and determine the user's condition and risk level. A machine learning engine may use algorithms to identify relationships between PC user activity and one or more other parameters, conditions, or states relevant to the collaboration session. In some embodiments, a rule-based engine may be used alone or in combination with other algorithms for determining user condition and the associated risk. After any health risk is determined, such as when the condit or risk level reach a threshold condition, the intelligence resource may generate an appropriate action.

In addition to being forwarded to the intelligence engine, user behavior information received in operation 302 (304) is used, in conjunction with historical user behavior information residing in intelligence engine 55, to evaluate the user's condition & risk level based on recent user behavior information. The illustrated method 300 further includes initiating a behavior-influencing action appropriate for the user's present condition, norms, and thresholds. If for example, the parameter of interest is the elapsed time during which the user's gaze point has remained on a display screen of the user's PC, and the elapsed time is approaching a value beyond which historical data indicates an observable increase in data entry errors, the illustrated method 300 may take a behavior-influencing action (310) by generating and sending a prompt to the PC user to take a break.

Importantly, the depicted method 300 does not terminate at the point of messaging or otherwise influencing the user's behavior. Instead, the illustrated method 300 include one or more post-message actions (312) referred to herein as extended actions. The extended actions explicitly illustrated in FIG. 3 include an evaluation action 320, which the user's post message behavior is tracked and mapped against various parameters of interest to determine an effectiveness of the initial action. Returning to the earlier example, if the user data obtained shortly after the user was prompted to take a break reveals that the users position has not substantially changed, the message's failure to motivate the user to action would be recorded and might result in the adoption of one or more additional or alternative actions. FIG. 3 further illustrates an extended action referred to as progressive action 332 and an extended action referred to as supplementary action 300.

Supplementary action 300 may include pushing a notification to a relative or coworker of the PC user or another similar action while progressive action may comprise sending a second or subsequent action with a higher probability of motivating a response from the user. As an example, an initial prompt make be in the form of a toast popup while a second action performed if the user does not respond, may consist of an audible and haptic alter.

Referring now to FIG. 4, a flow diagram illustrates a detailed implementation of a method 400 for assessing and influencing the behavior of a PC user to improve the user's wellbeing. The method 400 illustrated in FIG. 4 includes gathering (402) user data from available sources and transmitting the gathered data to two distinct infrastructures. Specifically, the user data gathered in operation 402 is sent (428) to a cloud infrastructure 398 and transmitted (404) to an edge infrastructure (399).

In the depicted implementation, cloud infrastructure 398 is configured to aggregate the user data in a cloud database 430 and feed (432) the user data to a machine learning engine where one or more machine learning algorithms are applied to the aggregated user data to develop a user data knowledge base, typically revealing one or more relationships between or among various parameters for which data has been collected. An inference engine 436 is applied to the user data knowledge base to deduce new information. The inference engine 434 illustrated in FIG. 4 may be used to score users against a set of one or more key performance indicators. User-specific rules are then updated (440) or, in the case of a new user, created based on the assigned KPI scores. A local database 444 within the edge infrastructure 399 is then updated (442).

Within the edge infrastructure 399, the illustrated process flow determines (406) whether rules exist for the current user. If no rules exist, the process flow transfers over to the cloud infrastructure 398, which will assign health KPI scores to the new user and stored the rules in local database 444. If the local database includes rules for the current user, the rules are read and the gathered information is evaluated to determine (412) whether the gathered data indicates one or more KPI changes. If no KPI change is detected, the process flow jumps to operation 402 and the method begins again. If, however, a KPI change is detected in block 412, the illustrated process flow determines (414) whether the gathered data is in compliance with the local rules. If so, process flow branches back to the KPI change detection operation 412. If, however, the gathered data is out of bounds with respect to the applicable rules, the health risk associated with the violation is determined (416) and an initial behavior-influencing action is taken (420). The process then determines whether (421) an extended action, as described above with respect to FIG. 3, is warranted and, if so, one or more extended actions is performed.

Although the present disclosure has been described in detail, it should be understood that various changes, substitutions, and alterations can be made hereto without departing from the spirit and the scope of the disclosure as defined by the appended claims.

What is claimed is:

1. A user wellness method, comprising:
receiving user behavior information pertaining to a personal computer (PC) user, from one or more smart devices associated with the user, wherein the personal computer is selected from a laptop, desktop, or hybrid information handling system;
aggregating collected user behavior information, learning user behavior norms and distributions, and defining one or more user behavior thresholds;
evaluating a user condition and risk level of the user based on recent user behavior data;
performing an initial behavior-influencing action appropriate for the user condition and risk level; and
performing one or more extended actions pertaining to the initial action.

2. The method of claim 1, wherein the one or more smart devices associated with the user include smart devices from a group comprising: a mobile device of the user, a PC of the user, and a wearable worn by the user.

3. The method of claim 1, wherein the initial behavior-influencing action comprises an electronic message delivered to one or more of the smart devices associated with the user.

4. The method of claim 3, wherein one of the one or more smart devices associated with the user comprises an active device and wherein the initial behavior-influencing action is delivered only to the active device.

5. The method of claim 3, wherein the electronic message comprises a reminder message selected from a group comprising a toast popup, a vibration, and an audible alert.

6. The method of claim 3, wherein the initial behavior-influencing action comprises auto scheduling for breaks, change of activity, and focus time.

7. The method of claim, 1, the one or more extended actions include evaluating an effectiveness of the initial behavior-influencing action.

8. The method of claim 7, further comprising: responsive to detecting an ineffective or under-effective behavior-influencing action, modifying the initial behavior-influencing action.

9. The method of claim 1, wherein the one or more extended actions include performing a progressive action, wherein the progressive action is at least one of: more intrusive or detectable, delivered to more of the user's smart devices, delivered two or more times, includes a more challenging behavior recommendation, and includes an express health and safety warning.

10. The method of claim 1, wherein the one or more extended actions include a supplementary action, wherein the supplementary action is indicative of the initial behavior-influencing action and is delivered to a recipient selected from: a family member or coworker of the user.

11. An information handling system, comprising:
a central processing unit (CPU) communicatively coupled to a system memory comprising executable instructions that, when executed by the CPU cause the CPU to perform wellbeing maintenance operations including:
receiving user behavior information pertaining to a personal computer (PC) user, from one or more smart devices associated with the user, wherein the personal computer is selected from a laptop, desktop, or hybrid information handling system;
aggregating collected user behavior information, learning user behavior norms and distributions, and defining one or more user behavior thresholds;
evaluating a user condition and risk level of the user based on recent user behavior data;
performing an initial behavior-influencing action appropriate for the user condition and risk level; and
performing one or more extended actions pertaining to the initial action.

12. The information handling system of claim 11, wherein the one or more smart devices associated with the user include smart devices from a group comprising: a mobile device of the user, a PC of the user, and a wearable worn by the user.

13. The information handling system of claim 11, wherein the initial behavior-influencing action comprises an electronic message delivered to one or more of the smart devices associated with the user.

14. The information handling system of claim 13, wherein one of the one or more smart devices associated with the user comprises an active device and wherein the initial behavior-influencing action is delivered only to the active device.

15. The information handling system of claim 13, wherein the electronic message comprises a reminder message selected from a group comprising a toast popup, a vibration, and an audible alert.

16. The information handling system of claim 13, wherein the initial behavior-influencing action comprises auto scheduling for breaks, change of activity, and focus time.

17. The information handling system of claim, 11, the one or more extended actions include evaluating an effectiveness of the initial behavior-influencing action.

18. The information handling system of claim 17, further comprising: responsive to detecting an ineffective or under-effective behavior-influencing action, modifying the initial behavior-influencing action.

19. The information handling system of claim 11, wherein the one or more extended actions include performing a progressive action, wherein the progressive action is at least one of: more intrusive or detectable, delivered to more of the user's smart devices, delivered two or more times, includes a more challenging behavior recommendation, and includes an express health and safety warning.

20. The information handling system of claim 11, wherein the one or more extended actions include a supplementary action, wherein the supplementary action is indicative of the initial behavior-influencing action and is delivered to a recipient selected from: a family member or coworker of the user.
